(19) United States
(12) Patent Application Publication
(10) Pub. No.: US 2021/0046139 A1
(43) Pub. Date: Feb. 18, 2021

(54) VEGETATION WATERS AND USES THEREOF

(71) Applicant: FATTORIA LA VIALLA DI GIANNI, ANTONIO E BANDINO LO FRANCO - SOCIETA' AGRICOLA SEMPLICE, Arezzo (IT)

(72) Inventors: Giuseppe LAURIA PINTER, Parma (IT); Roberto BIANCHI, Civiglio (Como) (IT); Daniele PIZZICHINI, Roma (IT); Gianni LO FRANCO, Arezzo (IT); Antonio LO FRANCO, Arezzo (IT); Bandino LO FRANCO, Arezzo (IT)

(73) Assignee: FATTORIA LA VIALLA DI GIANNI, ANTONIO E BANDINO LO FRANCO - SOCIETA' AGRICOLA SEMPLICE, Arezzo (IT)

(21) Appl. No.: 17/043,271

(22) PCT Filed: Mar. 26, 2019
(86) PCT No.: PCT/IB2019/052430

§ 371 Date: Sep. 29, 2020

(30) Foreign Application Priority Data
Mar. 30, 2018 (IT) 102018000004123

(51) Int. Cl. A61K 36/63; A61K 31/05; A61K 31/351; B01D 61/02; B01D 61/14; B01D 71/02; B01D 71/56

(52) U.S. Cl. CPC A61K 36/63; A61K 31/05; A61K 31/351; A61K 9/0053; B01D 61/147; B01D 71/025; B01D 71/56; B01D 61/025

(57) ABSTRACT

The present invention relates to a phytocomplex or natural concentrate rich in polyphenolic compounds such as hydroxytyrosol and 3,4-DHPA-EDA, derived from the waters from the pressing of olives for oil and/or olive pomace as residues of the olive milling process, for use in the reduction/attenuation of the symptoms and/or side effects associated with/caused by diabetes and/or the pathological conditions associated therewith.

The present invention relates to a natural phytocomplex rich in polyphenolic compounds, in particular rich in hydroxytyrosol and oleuropein aglycone (3,4-DHPA-EDA), derived from the waters from the pressing of olives for oil (commonly known as vegetation waters) and/or pomace oil residues of the olive milling process, for use in the reduction/attenuation of the symptoms and/or side effects associated with/caused by diabetes and/or the pathological conditions associated therewith.

### PRIOR ART

Diabetes is a multi-organ disease caused by an insulin deficiency due to a dysfunction of pancreatic beta cells and insulin resistance of the target organs.

Diabetes is the sixth cause of disability worldwide and leads to global health costs estimated at 825 billion dollars. In fact, due to the present-day lifestyle, the spread of diabetes is estimated as about 415 million people affected in 2015 and should increase to about 642 million by 2040. Furthermore, it is estimated that another 193 million people are affected by undiagnosed diabetes.

It has been demonstrated that an intensive management of glucose levels reduces the cardiovascular and cerebrovascular risks, in particular with reference to microvascular complications such as retinopathy, nephropathy and neuropathy, which represent the main causes of morbidity and mortality linked to diabetes.

Cataracts represent a further complication of diabetes which manifests itself in about 25% of diabetic patients. Its pathogenesis is closely correlated to chronic hyperglycemia and it is believed that a strict control of the levels of glycemia is fundamental in order to prevent the development and/or the progression thereof. Furthermore, one should not underestimate the fact that cataracts are one of the main causes of impaired vision in diabetic patients, who also pose particular complexities for the surgical approach to cataracts. Overall, cataracts represent a major health and economic problem, above all in developing countries, where the treatment of diabetes is insufficient and cataract surgery is often inaccessible.

Although the intensive control of glucose is by now a fairly well-established practice aimed at reducing the complications of diabetes, severe hypoglycaemia has been associated with a higher mortality at 12 months also in people who do not receive insulin. Safe, effective drugs, including insulin, reduce the level of glycemia by 1-2%.

The Mediterranean diet has been proposed as an important model for seeking to block or in any case reduce long-term diabetic complications and other metabolic syndromes such as obesity, atherogenic dyslipidaemia, hypertension and chronic low-grade inflammation.

It is well known, in fact, that olives and olive oil, important components of the Mediterranean diet, have a powerful antioxidant activity. Similarly, olive leaves contain substances with antioxidant properties that have been recently introduced into the European Pharmacopeia for the purpose of treating hypertension and diabetes.

There thus remains a strongly felt need to identify new beneficial effects correlated with the intake of olives and derivatives, in particular to improve public health and reduce and/or prevent the risks of widespread pathologies such as the ones described above.

The need is even more greatly felt when reference is made to waste products from olive processing, such as, for example, vegetation waters. In fact, although many studies on vegetation waters have been undertaken, there is still a greatly felt need to identify new properties that may lend value to these waste products, which would otherwise only be a cost for the producer and an environmental hazard. Particularly felt is the need to identify new nutritional and/or medical/pharmacological properties that may raise the value of this waste product.

In this regard, the Applicant proposes using vegetation waters for the purpose of treating diabetes and/or the pathological conditions associated therewith. In particular, the Applicant proposes using vegetation waters to reduce/attenuate the symptoms and/or side effects associated with/caused by diabetes and/or the pathological conditions associated therewith.

In fact, as reported in the example of the present application, the Applicant has demonstrated that the polyphenolic concentrate of the invention is effective, in particular, using a model of painful diabetic neuropathy, for the purpose of reducing the symptoms and/or conditions listed above, in addition to manifesting further positive effects that will be described in greater detail below.

In particular, the Applicant has demonstrated that the polyphenolic concentrate of the invention shows the following effects:
decrease in polyphagia, i.e. treatment with the concentrate of the invention induces a significant reduction in the consumption of food by animals with diabetes; and/or
decrease in polydipsia, i.e. treatment with the concentrate of the invention induces a significant reduction in the consumption of water by animals with diabetes; and/or
decrease in glycemia, i.e. treatment with the concentrate of the invention reduced the blood glucose concentration by about 8%; and/or
lowering of the level of glycated hemoglobin by about 24%; and/or
slowing of cataract progression; and/or
recovery of mechanical sensitivity, i.e. in diabetic rats a reduction in mechanical sensitivity of about 66% on average was observed; and/or
decrease in sensitivity to heat, i.e. in diabetic rats an increase in sensitivity to heat of about 70% on average was observed; and/or
restoration of the nerve conduction velocity compromised (slowed) as a result of the onset of diabetic pathology; i.e. treatment with the polyphenolic concentrate improves the motor and sensory nerve conduction velocity at the level of the sciatic nerve and tail by about 8-10%; and/or
reduction of about 20-25% in the concentration of thiobarbituric acid reactive substances (TBARS,) both in plasma and in the kidneys; and/or
reduction of about 39% in plasma triglyceride and cholesterol levels.

In conclusion, from the tests conducted by the Applicant it emerged that the symptoms and conditions associated with diabetes, in particular, diabetic neuropathy, improve considerably following treatment with the polyphenolic concentrate of the invention.

### BRIEF DESCRIPTION OF THE FIGURES

Further advantages of the present invention will be apparent from the detailed description and the appended figures, of which:

FIG. 1 shows the effects on the thermal nociceptive threshold of treatment with the polyphenolic concentrate or with HT on STZ diabetic rats and control rats. The STZ rats with induced diabetes show an increase in the thermal nociceptive threshold compared to the controls. The administration of the polyphenolic concentrate is capable of decreasing the nociceptive threshold, whereas the administration of HT does not induce a significant reduction.

FIG. 2 shows the effects on the mechanical nociceptive threshold of treatment with the polyphenolic concentrate or with HT on STZ diabetic rats and on the control rats. The STZ rats with induced diabetes show a decrease in the mechanical nociceptive threshold compared to the controls. The administration of the polyphenolic concentrate and of HT is capable of increasing the mechanical nociceptive threshold.

FIG. 3 shows the effects on cataract development of treatment with the polyphenolic concentrate and with HT on the diabetic rats and on the controls. The rats with diabetes show the onset of cataract at the ninth week after the treatment. Treatments both with the concentrate and with HT reduce and prevent the onset of cataracts.

### DEFINITIONS

In the context of the present invention, glycemia means the concentration of glucose in the blood.

In the context of the present invention, diabetes means a chronic disease classifiable in the group of pathologies known as diabetes mellitus, characterized by a high concentration of glucose in the blood, in turn caused by an insulin deficiency (absolute or relative) in the human body and/or an altered use thereof (insulin resistance). Diabetes is characterized by polyuria (abundant urine production), polydipsia (abundant water intake) and polyphagia (excessive hunger).

In the context of the present invention, diabetic neuropathy means damage to and/or malfunctioning of the nervous system caused by diabetes, in particular the nerve fibers that are responsible for transmitting information from the brain to different parts of the body.

In the context of the present invention, painful diabetic neuropathy means a neuropathy due to diabetes correlated with chronic pain in one or more regions of the human body.

In the context of the present invention, diabetic nephropathy means a functional and/or structural reduction in kidney capacity.

Furthermore, in the context of the present invention, cataract means an opacification of the crystalline lens, whose severity is assessed according to a semi-quantitative scale that attributes to each eye the stages of opacification described in the Example.

### DETAILED DESCRIPTION OF THE INVENTION

The present invention relates to a phytocomplex or concentrate of vegetation waters and/or olive pomace comprising polyphenolic compounds, preferably hydroxytyrosol and 3,4-DHPA-EDA, for use in the treatment and/or prevention of diabetes and/or a pathology and/or complication associated with/caused by diabetes.

Hereinafter reference will be made to this phytocomplex or concentrate simply as “concentrate” or “polyphenolic concentrate”.

A further aspect of the present invention relates to a composition comprising the concentrate and further excipients/ingredients that are pharmacologically accepted for use in the treatment and/or prevention of diabetes and/or a pathology and/or complication associated with/caused by diabetes.

According to a preferred embodiment of the invention, the concentrate and/or composition are particularly indicated for the purpose of reducing/attenuating/improving the symptoms associated with diabetes and/or said pathology/complication associated with/caused by diabetes.

Therefore, according to a preferred aspect of the invention, the concentrate and/or composition is(are) indicated for the treatment and/or prevention and/or reduction of the symptoms and/or side effects associated with/caused by diabetes and/or a pathology/complication associated with/caused by diabetes.

The diabetes is preferably type 1 (insulin-dependent) or type 2 (non-insulin-dependent).

Said pathology/complication associated with/caused by diabetes is preferably selected from: diabetic neuropathy, preferably painful diabetic neuropathy, nephropathy, alteration of pancreatic function/structure, retinopathy, diabetic foot and dry skin with vasomotor disautonomic manifestations.

Said symptoms and/or said side effects are preferably selected from: reduction of hyperglycemia and/or elevated levels of glycated hemoglobin, onset and/or development/progression of cataracts and retinopathy, and alterations in thermal and/or mechanical sensitivity.

In some cases, said pathology/complication associated with/caused by diabetes can also be a symptom and/or side effect associated with/caused by diabetes.

In a further preferred embodiment of the invention, the concentrate and/or composition is(are) preferably used to improve the motor and/or sensory nerve conduction velocity, which is preferably reduced in subjects affected by this pathology.

The vegetation waters are preferably derived from a three-phase (oil, vegetation water and pomace), and/or a two-phase (oil and pomace+vegetation water) olive milling process. The vegetation waters generated by the mill can preferably be treated with an acidic pH solution, preferably at a pH ranging from 3 to 5, preferably 4/5, for example, by adding a strong acid, and/or pectolytic enzymes, i.e. enzymes that hydrolyze the cellulosic matrix of olive skins.

The olive pomace is preferably pitted, diluted and/or prefiltered. The olive pomace preferably has a particle size or cut-off ranging from 0.5 to 1 millimeters (mm), more preferably about 0.7 mm. An example of a particle size is the one obtained by sieving with a vibrating screen.

The pitted olive pomace can be solubilized or dispersed in an aqueous matrix with a pH comprised from 3 to 5, preferably from 3.5 to 4.0.

The solubilization step has the purpose of solubilizing the polyphenols that would otherwise remain trapped in the solid matrix of the olive skins.

In a preferred embodiment of the invention, the concentrate can further comprise: at least one further phenolic compound preferably selected from: tyrosol, chlorogenic acid, β-hydroxyverbascoide, rutin, verbascoide, and luteolin; and/or at least one metal preferably selected from: sodium, calcium, magnesium and potassium; and/or at least one anion preferably selected from: chlorides, sulphates, phosphates and nitrates; and/or at least one carbohydrate selected from: glucose, fructose, mannitol and sucrose.

In a further embodiment of the invention, the concentrate can comprise nitrogenous substances (proteins, amino acids), preferably in an amount comprised from 15 to 60 mg/kg, more preferably from 20 to 40 mg/kg (mg of nitrogen per liter of active solution).

In any case, the phenolic compounds present in the concentrate in the largest amount are hydroxytyrosol and 3,4-DHPA-EDA.

The amount of hydroxytyrosol preferably ranges from 1 to 10 grams per liter of vegetation waters (g/L), more preferably from 1.5 to 5 g/L, even more preferably from 2 to 3 g/L.

The amount of 3,4-DHPA-EDA is preferably comprised from 0.5 to 8 g/L, more preferably from 1 to 6 g/L, even more preferably from 1.5 to 2.5 g/L. The amount of tyrosol is preferably comprised from 0.1 to 0.4 g/L, more preferably from 0.15 g/L to 0.25 g/L.

The amount of chlorogenic acid is preferably comprised from 0.06 to 0.24 g/L, more preferably from 0.8 to 0.16 g/L.

The amount of β-hydroxyverbascoide is preferably comprised from 0.3 to 1.5, more preferably from 0.5 to 1 g/L.

The amount of rutin is preferably comprised from 0.05 to 0.2 g/L, more preferably from 0.08 to 0.15 g/L.

The amount of verbascoside is preferably comprised from 0.4 to 1.7 g/L, more preferably from 0.6 to 1 g/L.

The amount of luteolin is comprised from 0.1 to 0.5 g/L, more preferably from 0.15 to 0.28 g/L.

The amount of sodium is preferably comprised from 75 to 300 mg/L, more preferably from 120 to 180 mg/L.

The amount of calcium is preferably comprised from 5 to 10 g/L, more preferably from 2 to 5 g/L.

The amount of magnesium is preferably comprised from 220 to 900 mg/L, more preferably from 400 to 500 mg/L.

The amount of potassium is preferably comprised from 3 to 15 g/L, more preferably from 6 to 9 g/L.

The amount of chlorides is preferably comprised from 1.5 to 7 g/L, more preferably from 2.5 to 4.5 g/L.

The amount of sulphates is preferably comprised from 12 to 45 g/L, more preferably from 18 to 28 g/L.

The amount of phosphates is preferably comprised from 1.5 to 7 g/L, more preferably from 2.5 to 5 g/L.

The amount of nitrates is preferably comprised from 12 to 50 mg/L, more preferably from 18 to 30 mg/L.

The amount of glucose is preferably comprised from 15 to 60 g/L, more preferably from 25 to 35 g/L.

The amount of fructose is preferably comprised from 3.5 to 15 g/L, more preferably from 5 to 9 g/L.

The amount of mannitol is preferably comprised from 1 to 4 g/L, more preferably from 1.5 to 3 g/L.

The amount of sucrose is preferably comprised from 4 to 16 g/L, more preferably from 6 to 10 g/L.

In a preferred embodiment of the invention, the concentrate is obtained/obtainable by means of a process comprising the steps of: (i) microfiltering a sample of the vegetation waters and/or olive pomace so as to obtain a concentrate and a permeate of microfiltration; and (ii) concentrating by reverse osmosis the microfiltration permeate obtained from step (i).

The microfiltration is preferably performed after the solubilization step as described before.

The microfiltration has the purpose of separating a concentrate, i.e. the concentrated fraction of the content of the vegetation waters/olive pomace in suspension, for example micro fragments, fibers and corpuscular material such as cells and bacteria. It is carried out under the standard conditions for this type of matrix.

Following the microfiltration step, in addition to the concentrate, one obtains a permeate, i.e. a clear fraction, characterized by a color that varies according to the starting material and contains the dissolved components of the vegetation waters/olive pomace, e.g. proteins, sugars, salts, polyphenols, organic acids and various soluble organic molecules.

The microfiltration is preferably carried out with at least one, preferably two, ceramic membrane(s). The membrane is characterized by a preferably tubular shape.

In a preferred embodiment, the membrane is made of alumina oxide and/or zirconia.

The membrane preferably has the following characteristics: an outer diameter ranging from about 30 to about 40 mm, preferably of about 25 mm; and/or a length ranging from about 500 to about 1500 mm, preferably of about 1200 mm; and/or a series of channels with a diameter, preferably a hydraulic diameter, ranging from about 2.5 to about 5 mm, preferably of about 3.5 mm; and/or a filtering surface ranging from about 0.15 to about 0.7 m², preferably of about 0.35 m²; and/or a particle size or molecular weight cut-off ranging from about 0.1 micron to about 300 kDa.

More preferably, the membrane has all of the characteristics stated above. The reverse osmosis step for concentrating the permeate obtained from the microfiltration of the vegetation waters/olive pomace as described before is carried out under the standard conditions for this type of matrix, preferably by using a polymeric membrane, more preferably made of polyamide.

In particular, the membrane has a spiral-wound conformation and/or a molecular weight cut-off with high salt rejection, i.e. capable of rejecting sodium chloride molecules at a percentage of 99.9%. This means that the osmosis membrane holds back the molecules of biomedical interest and allows only water molecules to pass through.

The polymeric membrane preferably has a filtering surface ranging from about 5 to about 15 m², more preferably of about 7 m².

The reverse osmosis step enables the permeate obtained by microfiltration to be concentrated preferably by about 4 times; this means that from 100 L of microfiltration permeate 25 L of concentrate are obtained.

In this case the volume concentration ratio (VCR) is 4, i.e. 100/25.

The VCR can change based on the starting matrix (vegetation waters) and above all based on its salt content, because the reverse osmosis process must offset the osmotic pressure of the matrix which is going to be concentrated.

The present invention further relates to a concentrate (or phytocomplex) of vegetation waters/olive pomace obtainable/obtained with the above-described process.

The polyphenolic concentrate preferably has the composition described before as regards the content of phenolic compounds, metals, carbohydrates, anions and nitrogen.

According to a further aspect of the invention, the concentrate and/or composition as described above is/are used alone or in combination with other substances, compounds, drugs or compositions with a protective and/or curative action against diabetes, such as insulin, oral hypoglycemizing agents, other substances with antidiabetic pharmacological activity, either known or of potential interest and undergoing study, as well as in combination with physical activity.

The concentrate of vegetation waters and/or olive pomace and/or the composition for the above-described uses is(are) preferably formulated for oral administration, preferably as a drink. The drink according to the invention can further comprise one or more optional excipients normally present in this type of formulation.

The drink can preferably be water- and/or fruit- and/or milk-based. In a particularly preferred embodiment of the invention, the drink is fruit-based, preferably grape-based, preferably grape juice and/or must, more preferably organic grapes.

Alternatively, the concentrate of vegetation waters and/or olive pomace and/or the composition for the above-described uses is(are) formulated as lozenges, pills, capsules, tablets or the like.

In other words, by virtue of the therapeutic effects found and described herein, the drink and/or the oral formulation can be taken as a dietary supplement, preferably for the purpose of treating and/or preventing diabetes and/or a pathology and/or complication associated with/caused by diabetes, as previously described, or for the treatment of the symptoms and/or side effects associated with/caused by diabetes and/or said pathology and/or complication associated with/caused by diabetes, as previously described.

The drink and/or the oral formulation can optionally be taken in association with one or more further substances, compounds, drugs or compositions useful for that purpose.

Alternatively, the concentrate of vegetation waters and/or olive pomace and/or the composition for the above-described uses is(are) formulated for topical application, preferably as a: cream, oil, ointment, aerosol, gel, vaginal suppository, spray, solution, patch, gauze, bandage, granules or powder. Said formulation, preferably when used topically, is preferably indicated for the treatment of the symptoms and/or side effects associated with/caused by diabetes and/or said pathology and/or complication associated with/caused by diabetes as previously described, preferably selected from: diabetic foot and dry skin with vasomotor disautonomic manifestations.

The formulation for topical use described above optionally further comprises agents/molecules with a biologically active function preferably selected from: cicatrizing, anti-inflammatory, antibiotic, emollient, soothing, analgesic and combinations thereof.

The considerable advantage tied to the use of the polyphenolic concentrate of the invention compared to hydroxytyrosol as such, besides a more appreciable improvement of several symptoms or conditions of diabetes and diabetic neuropathy, also lies in its inexpensiveness. The concentrate is in fact obtainable from vegetation waters and/or olive pomace, which represent a waste material of the oil industry and, being an environmental pollutant, must be properly disposed of, resulting in considerable costs.

The possibility of using such waste materials to obtain, by means of simple, inexpensive processes that do not require complex equipment or instruments, an effective product that may be used in the fields of pharmaceutics, nutraceutics and food additives, makes the polyphenolic concentrate a valid alternative to the use of the drugs presently used for the treatment of diabetes and/or a pathology and/or complication associated with/caused by diabetes as previously described, in particular, neuropathy, which, as has been seen, are associated with considerable undesirable effects, as well as to the use of pure hydroxytyrosol.

### Example

Pharmacological Treatment The quantification of the presence of polyphenols in the concentrate was carried out by HPLC (high-performance liquid-chromatography). The results demonstrate that the polyphenol present in the largest amount is hydroxytyrosol (HT), present at 5.5 g/I. Pure hydroxytyrosol was supplied by Macker Chemie Italia (Milan). The dosage of HT and of the concentrate of the present invention is 50 mg/kg.

Animals

Use was made of 48 male Sprague-Dawley rats (Harlan, Italy) with a weight comprised from 180-200 g.

Induction of Diabetes and Experimental Treatments

A well-established model of streptozotocin (STZ)-induced diabetes in rats was used. Streptozotocin is a glucosamine derivative of nitrosurea which selectively destroys the β cells of the pancreatic islets and causes the development of diabetes with hyperglycemia and glycosuria. The model shares a series of characteristics with human diabetic complications at both a functional level and a biochemical level, such as, for example, a reduced nervous conduction, loss of small-diameter sensory nerve fibers in the skin, reduction in Na+ and K+-ATPase activity and early alterations of the thermal and mechanical nociceptive thresholds.

In this study, diabetes was induced in 24 rats with a single intraperitoneal injection of 60 mg/kg of STZ dissolved in a sodium citrate buffer (pH 4.5). 24 rats injected only with the vehicle were used as non-diabetic controls. Hyperglycemia was confirmed by measuring glycosuria 72 hours after the injection of STZ using Keto-Diabur strips (KD-5000; Roche Diagnostics Spa, Italy). Only the animals with glycosuria >5% were classified as diabetic and included in the study. One rat did not meet the criteria and was excluded from the trial.

The animals were then randomized to receive the concentrate of the invention at an HT equivalent dose of 50 mg/kg/day or 50 mg/kg/day of pure HT in drinking water, according to the following treatment groups:

(1) non-diabetic controls treated with the vehicle (n=8);

(2) non-diabetic controls treated with the concentrate according to the present invention, HT equivalent 50 mg/kg/day (n=8);

(3) non-diabetic controls treated with HT, 50 mg/kg/day (n=8);

(4) diabetics (due to STZ) treated with the vehicle (n=8);

(5) diabetics (due to STZ) treated with the concentrate according to the present invention, HT equivalent 50 mg/kg/day (n=8);

(6) diabetics (due to STZ) treated with HT, 50 mg/kg/day (n=7);

The daily dose of compounds was administered starting from the 7th week after the injection of STZ or the vehicle. The treatment lasted 5 weeks and urine samples were taken. The animals were then sacrificed and the plasma, kidneys and sciatic nerves were removed. The sacrifice took place within 2 hours after the last administration of the concentrate, HT or the vehicle.

During the trial, the rats were weighed weekly to monitor their weight gain/loss. Hyperglycemia was confirmed in every rat by measuring the glucose level in the blood taken from the vein of the tail with a suitable apparatus (Glucomen, Menarini, Italy), 15 days after the injection of STZ. A plasma glucose level above 300 mg/dL was defined as the hyperglycemia threshold for defining the animals as diabetics.

Thermal Nociceptive Threshold

The nociceptive threshold for radiant heat was quantified using the hot plate test. A 40 cm long plexiglass cylinder was placed on the hot plate (Ugo Basile, Varese, Italy) to contain the animal; the plate temperature was maintained at 50±0.2° C. The latency time was defined as the time elapsing between the positioning of the rat on the hot plate and the moment of hind paw withdrawal or licking, or the discomfort manifested by the animal. The test was performed 15 and 30 days after the injection of STZ or of the vehicle, one week after the administration of the concentrate of the invention and prior to sacrifice. Every animal was tested twice; the tests were separated by a 30-minute rest interval and the values were averaged.

Mechanical Nociceptive Threshold

The mechanical nociceptive threshold (Randall-Selitto test) was assessed using an electromechanical apparatus (Ugo Basile, Varese, Italy). This instrument generates an increasing linear mechanical force applied directly on the dorsal surface of the rat's hind paw by means of a cone-shaped piston. The results represent the maximum pressure (expressed in grams) tolerated by the animals as manifested with the withdrawal of the paw. The test was performed at the times indicated for the determination of the thermal threshold (see above). In the course of every measurement test the animal was tested twice, with a rest interval of 30 minutes between one measurement and the other, and the values are the average of the two determinations.

Eye Examination and Cataract Classification

The opacity of the eyes and of the crystalline lenses was examined every week starting from the seventh/eighth week after the induction of diabetes, the period in which, in the STZ diabetic model, cataracts manifest themselves. Cataract severity was classified using a validated semi-quantitative scale. In particular, the following assessment scheme and the following scores were used:

| | Score |
|---|---|
| Phase 0: crystalline lenses transparent | 0 |
| in both eyes | |
| Phase 1: crystalline lens transparent | 1 |
| in one eye, slightly opaque in the | |
| other eye | |
| Phase 2: both the crystalline lenses | 2 |
| slightly opaque | |
| Phase 3: crystalline lens with a mature | 3 |
| cataract in one eye, slightly opaque | |
| in the other eye | |
| Phase 4: both crystalline lenses with | 4 |
| a mature cataract | |

The opacity index was calculated to quantitatively assess the degree of opacity of the crystalline lens by means of the following formula:

$$Opacity index = \frac{\begin{matrix}number of eyes in each phase \times \\ stage of the eye\end{matrix}}{Total number of eyes}$$

Motor and Sensory Conduction Velocity

The electrophysiological studies were performed using a portable electromyograph (Alpine Biomed ApS DK-2740, Denmark). The test on sciatic nerve conduction was performed by stimulating the sciatic nerve in the Achilles tendons and in the hollow above the sciatic nerve at the point of attachment of the tail with a single 2.2 ms supramaximal pulse using a bipolar electrode. A measurement of the motor action potential (CMAP) was obtained by placing the active electrode in subcutaneous tissue, in the first interosseous muscle of a toe of the hind paw, and the reference electrode in the fourth interosseous muscle of the same paw. The motor conduction velocity was calculated by subtracting the distal value from the proximal value taking the latency of the first negative peak of the proximal CMAP, measured in milliseconds, and the difference was divided by the distance between the two stimulating electrodes, measured in millimeters.

The sensory conduction velocity was determined using the plantar nerve. The sensory nerve action potential (SNAP) of the plantar nerve was measured by placing the recording electrode (needle) in subcutaneous tissue at the level of the hip next to the medial malleolus and the reference electrode proximally at about 1 cm. Circular stimulation electrodes were placed in the middle of the three middle toes of the hind paw, and stimulation was performed with a square pulse wave lasting 0.05 ms, with a barely supramaximal current intensity stimulus.

The sensory conduction velocity was calculated by measuring the latency at the negative deviation of the peak potential and the distance between the stimulation electrode and the recording electrode. The motor and sensory conduction velocities are expressed as meters per second.

The tail motor and sensory conduction test requires that the sensory nerve of the tail be stimulated by inserting the cathode 2 cm from the tip of the tail and the anode at 1 cm distally, using a square wave pulse lasting 0.05-ms at a supramaximal intensity. The SNAPs were recorded by inserting the active electrode 5 cm from the cathode and the reference electrode at 1 cm more proximally. The sensory nerve conduction velocity was calculated by measuring the latency at the peak of the first negative deflection and the distance between the stimulating and recording electrodes.

The tail motor nerve conduction was obtained using a bipolar recording configuration. The active electrode was positioned 2 cm from the tip of the tail and the reference electrode at 1 cm distally. The motor nerve of the tail was initially stimulated at 5 cm proximally from the active recording electrode and, subsequently, proximally at 10 cm. The motor nerve conduction velocity was calculated by subtracting the proximal CMAP from the distal CMAP, starting from the first negative peak, measured in milliseconds; the difference was divided by the distance between the two stimulating electrodes, measured in millimeters.

Thiobarbituric Acid Reactive Substances (TBARS)

At the end of the 5-week treatment period, plasma and kidneys were collected. EDTA and glutathione were added to the plasma and kidney homogenate with final concentrations of 1.34 and 0.65 mmol/L, respectively. The levels of TBARS were determined as an indicator of the production of reactive oxygen species. 100 μL of plasma or of kidney homogenate were boiled in 0.6 mL of 1% phosphoric acid (w/v) and 0.2 mL of thiobarbituric acid (0.42 mmol/L) for 45 min. The mixture was cooled, and then an extraction was performed by stirring it with 1.2 ml of n-butanol and separating the two phases by centrifugation (10-20 min at 1500 g). The upper phase was measured using fluorimetry, (Infinite M200; Tecan, Milan, Italy) at an excitation wavelength of 532 nm and emission wavelength of 553 nm. The calibration curve was prepared with the standard 1,1,3,3,3-tetraethoxypropane at a final concentration of 1.64 μmol/mL.

Other Biochemical Analyses

At the end of the 5-week treatment period, urine and plasma were collected to measure the level of triglycerides (TG), total cholesterol (TC), high-density lipoprotein (HDL) and other parameters using commercially available diagnostic kits.

Statistical Analysis

The data were analyzed by means of two-way analysis of variance (ANOVA), with the treatment and the disease as independent variables, followed by the Student-Newman-Keuls post-hoc test. The data are reported as the mean±SEM. A two-tailed test value of p<0.05 was considered significant. All of the analyses were performed with StatView 5 (SAS Institute Inc.).

Changes in Body Weight, Daily Food and Water Intake, Plasma Glucose and Glycated Hemoglobin (HbA1c)

The untreated diabetic rats had a lower body weight than the control rats and the administration of both the concentrate and HT in the diabetic groups did not change this situation. The treatment of non-diabetic control rats with the concentrate or with HT did not show an increase in weight compared to the untreated control rats (Table 1a). A 40% increase in food intake was observed in the diabetic rats treated with STZ compared to the controls. At the end of the 5 weeks of treatment, the group treated with the polyphenolic concentrate—but not the group treated with HT—was capable of countering 25% of the polyphagia (Table 1a). Similarly, an increase in the daily water intake in the group treated with STZ was reduced in the group treated with the polyphenolic concentrate (Table 1a; reduction of polydipsia).

The blood glucose and HbA1c values increased significantly in untreated diabetic rats. HbA1c was significantly reduced in both groups treated with the polyphenolic concentrate and with HT, by 24% and 20% respectively (p<0.01 vs. the group not treated with STZ). The treatment of the control rats with the concentrate and with HT did not influence the glycemia or HbA1c values.

TABLE 1a

| | BODY | FOOD | WATER |
|---|---|---|---|
| | WEIGHT | INTAKE | INTAKE |
| GROUP | (g) | (g/24 h) | (ml/24 h) |
| CTRL | 459.9 ± 10.7 | 21.9 ± 0.8 | 60.8 |
| (n = 8) | | | |
| CTRL + concentrate | 456.0 ± 9.1 | 20.1 ± 0.4 | 80.6 |
| (n = 8) | | | |
| CTRL + HT | 450.4 ± 16.4 | 20.6 ± 0.9 | 69.5 |
| (n = 8) | | | |
| STZ | 212.7 ± 12.7** | 35.5 ± 2.0 * | 180.4 * |
| (n = 12) | | | |
| STZ + concentrate | 212.6 ± 8.7** | 28.2 ± 0.8 | 161.3 * |
| (n = 10) | | | |
| STZ + HT | 216.6 ± 13.0** | 33.3 ± 3.7 | 170.1* |
| (n = 9) | | | |

TABLE 1b

| | PLASMA | |
|---|---|---|
| | GLUCOSE | HbA1c |
| GROUP | (mg/dl) | (mM/mol) |
| CTRL | 214.0 ± 5.5 | 17.0 ± 0.6 |
| (n = 8) | | |
| CTRL + | 189.9 ± 6.7 | 16.7 ± 0.2 |
| concentrate | | |
| (n = 8) | | |
| CTRL + HT | 186.1 ± 3.2 | 16.7 ± 0.2 |
| (n = 8) | | |
| STZ | 789.8 ± 28.0 **** | 69.0 ± 2.9 **** |
| (n = 12) | | |
| STZ + | 746.0 ± 21.7**** | 53.9 ± 3.2 ****§§ |
| concentrate | | |
| (n = 10) | | |
| STZ + HT | 729.6 ± 36.5**** | 52.3 ± 5.3 ****§§ |
| (n = 9) | | |

Tables 1a and 1b summarize the values as the mean±S.E. per number (n) of rats per group. Comparisons were made with the one-way ANOVA test. p<0.05 vs. CTRL, CTRL+concentrate or CTRL+HT; ** p<0.01 vs. CTRL, CTRL+concentrate or CTRL+HT; *** p<0.005 vs. CTRL, CTRL+concentrate or CTRL+HT; **** p<0.001 vs. CTRL, CTRL+concentrate or CTRL+HT; §§ p<0.01 vs. STZ.

Changes in Kidney and Plasma TBARS, Total Cholesterol (TC), High Density Lipoprotein (HDL) and Triglycerides (TG)

STZ-induced diabetes significantly increases kidney and plasma TBARS (86 and 57%, respectively, p<0.05). The treatment with the concentrate reduced the plasma levels of TBARS by 18%. As shown in Table 2, the levels of TC, HDL and TG increased significantly in the diabetic rats compared to the non-diabetic rats (P<0.001). The administration of the concentrate significantly reduced the plasma levels of TC (P<0.001), TG (P<0.05) and LDL (P<0.05) compared to the STZ diabetic group. Table 2 below summarizes the effects of the diet supplemented with the concentrate of the present invention and with HT on plasma and kidney TBARS, and on TC, HDL, and TG of rats in the different experimental groups at the end of the administration.

TABLE 2

| | PLASMA | KIDNEY | | | |
|---|---|---|---|---|---|
| | TBARS | TBARS | PLASMA | PLASMA | PLASMA |
| | mmoles/ml | mmoles/ml | TC | HDL | TG |
| GROUPS | MDA | MDA | (mg/dl) | (mg/dl) | (mg/dl) |
| CTRL | 7.3 ± 0.4 | 16.9 ± 2.3 | 89.0 ± 4.2 | 26.7 ± 0.6 | 112.7 ± 6.7 |
| n = 8 | | | | | |
| CTRL + | 5.1 ± 0.6 | 12.8 ± 1.7 | 89.3 ± 2.9 | 26.1 ± 0.8 | 101.3 ± 7.3 |
| concentrate | | | | | |
| n = 8 | | | | | |
| CTRL + HT | 4.3 ± 0.5 | 19.1 ± 1.7 | 84.4 ± 2.1 | 27.0 ± 0.4 | 111.5 ± 8.6 |
| n = 8 | | | | | |
| STZ | 13.6 ± 1.1* | 26.6 ± 2.6*^{∘∘} | 125.7 ± 5.9**** | 49.2 ± 2.0**** | 503.8 ± 53.3**** |
| n = 12 | | | | | |
| STZ + | 10.8 ± 0.9 | 19.9 ± 2.6 | 95.1 ± 5.0 §§§§ | 38.5 ± 2.1****§ | 307.0 ± 50.9 § |
| concentrate | | | | | |
| n = 10 | | | | | |
| STZ + HT | 12.1 ± 1.2 | 18.6 ± 3.1 | 118.1 ± 3.0 | 48.7 ± 1.6**** | 457.1 ± 60.7**** |
| n = 9 | | | | | |

The values represent the mean±S.E per n rats per group. A comparative analysis was performed with the one-way ANOVA test. * p<0.05 vs. CTRL, CTRL+concentrate or CTRL+HT; **** p<0.001 vs. CTRL, CTRL+concentrate or CTRL+HT; ºº p<0.01 vs. CTRL+concentrate; § p<0.05 vs. STZ; §§§§ p<0.001 vs. STZ

Effects of STZ-Induced Diabetes, the Administration of the Concentrate and HT on the Thermal Nociceptive Threshold

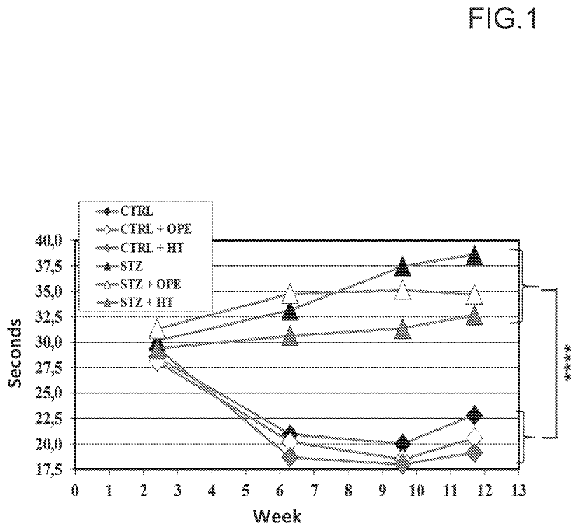

STZ-induced diabetes significantly influenced the thermal nociceptive threshold. At the end of the 5-week period, the diabetic rats showed a thermal nociceptive threshold that was 69% higher compared to the non-diabetic controls (FIG. 1, p<0.001). The administration of HT reduced the impairment of the thermal nociceptive threshold in diabetic rats without reaching statistical significance. At the end of the 5-week treatment, the group treated with the polyphenolic concentrate showed the lowest threshold compared to untreated STZ rats.

Effects of STZ-Induced Diabetes and the Administration of the Concentrate and HT on the Mechanical Nociceptive Threshold

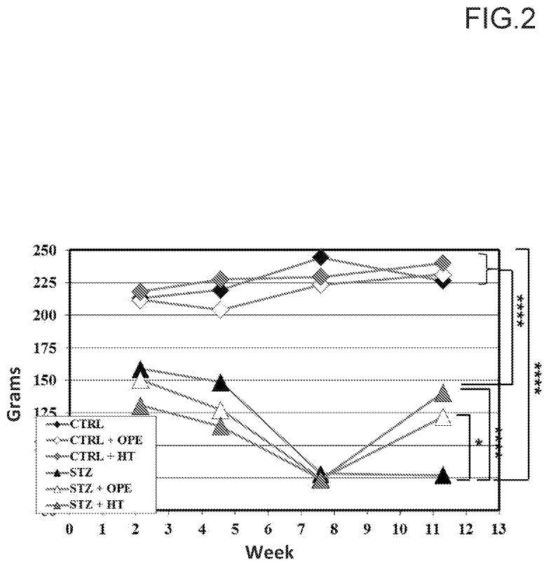

STZ-induced diabetes significantly influenced mechanical nociception (FIG. 2). At the end of the 5 weeks of treatment, the threshold was reduced by 66% in the diabetic animals compared to the non-diabetic ones. The administration both of the concentrate and HT had a significant effect on the mechanical nociceptive threshold (p<0.001). Furthermore, there were significant differences in the values of the Randall-Selitto test between the un-treated STZ group and the diabetic rats treated with the concentrate and with HT. The administration of the concentrate and of HT had no effect on the mechanical nociceptive threshold in the non-diabetic rats.

Effects of STZ-Induced Diabetes and the Administration of the Concentrate and HT on Cataract Development and Progression

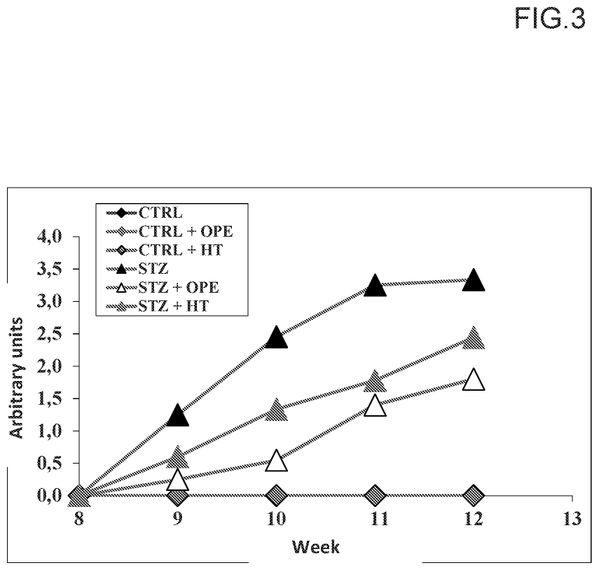

Cataract development was assessed once a week for 13 consecutive weeks starting from the eighth week after the induction of diabetes. FIG. 3 shows the score in the experimental groups. The crystalline lenses of the control rats obtained a score of 0 throughout the trial period. Cataract onset occurred at the 9th week in all groups. The severity of the score increased during the study period in the untreated STZ group, with maximum opacification (score of 3.7) at the 12th week. The administration of the concentrate of the present invention or of HT reduced cataract severity (FIG. 3). At the end of the fifth week, 67% of the untreated diabetic animals were in the fourth cataract phase, whereas only 30% of the diabetic rats treated with the concentrate or with HT were in the fourth cataract phase. The opacity index at the end of treatment was 5.3 in the untreated diabetic rats, 2.0 in the diabetic rats treated with HT and 1.6 in the diabetic rats treated with the concentrate.

Furthermore, at the end of the treatments, the group treated with the concentrate still had 7 eyes with cataracts at stage 0; the group treated with HT also had 3 eyes at stage 0, whereas the untreated diabetic group had no eyes at stage 0.

Overall, these data strongly suggest that the concentrate delays the onset and slows the progression of diabetic cataracts more effectively even than HT.

Effects of STZ-Induced Diabetes and the Administration of the Concentrate and of HT on the Tail and Sciatic Nerve Conduction Velocity (NCV)

The tail motor and sensory conduction velocities had decreased significantly at 5 weeks in the diabetic rats treated with the vehicle compared to the control rats. The treatment with the concentrate or with HT significantly improved the tail NCV compared to untreated diabetic rats (Table 3).

Table 3 below summarizes the effects of the treatment with the polyphenolic concentrate and with HT on the NCV of the sciatic nerve and tail (NCV) of the rats in the various experimental groups at the end of administration (week 12).

TABLE 3

| | | | Conduction of | Sensitivity |
|---|---|---|---|---|
| | Conduction | Sensitivity | sciatic | of sciatic |
| | of tail | of tail | nerve | nerve |
| GROUPS | (m/sec.) | (m/sec.) | (m/sec.) | (m/sec.) |
| CTRL | 39.4 ± 0.5 | 35.5 ± 0.6 | 49.2 ± 2.2 | 39.4 ± 1.5 |
| n = 8 | | | | |
| CTRL + | 34.7 ± 2.2 | 35.3 ± 1.3 | 47.7 ± 0.9 | 38.8 ± 0.6 |
| Concentrate | | | | |
| n = 8 | | | | |
| CTRL + HT | 36.1 ± 1.7 | 34.7 ± 1.3 | 46.5 ± 1.5 | 39.0 ± 0.9 |
| n = 8 | | | | |
| STZ | 34.2 ± 1.2 | 28.8 ± 0.8**** | 40.6 ± 1.1**** | 32.1 ± 0.8**** |
| n = 12 | | | | |
| STZ + | 37.0 ± 1.8* | 30.3 ± 0.8* | 43.0 ± 0.9* | 35.0 ± 0.8* |
| Concentrate | | | | |
| n = 10 | | | | |
| STZ + HT | 37.4 ± 0.9 | 30.8 ± 0.8 **** | 40.3 ± 0.7**** | 33.7 ± 0.9**** |
| n = 9 | | | | |

The values are the mean±S.E. Comparisons were made with the one-way ANOVA test. * p<0.05 vs. CTRL groups; **** p<0.001 vs. CTRL groups.

During the 5-week treatment period, the control rats showed the expected increase in NCV, about 10%, in m/s. The diabetic rats, by contrast, showed no increase. At the end of the 5-week period, the diabetic rats treated with the vehicle had a reduction in NCV of about 18% compared to the control rats. The rats treated with the concentrate or with HT had NCV values that were 13-25% higher. Neither the concentrate nor HT showed any effect on the NCV values in non-diabetic rats.

### CONCLUSIONS

A unique characteristic of the present example is the use of a 12-week therapeutic protocol in which the treatment with the concentrate and with HT was started after the onset of diabetes and with experimentally documented complications in order to provide data that faithfully reproduced the human condition.

The rats treated with the concentrate of the present invention showed a 22-24% reduction in HbA1c compared to the untreated diabetic rats, corresponding to a 1.5% decrease in the HbA1c level, from 8.5% to 7.0%. The considerable relevance of these data regards the correlation between the control of diabetic hyperglycemia and the reduction of the incidence of macrovascular and microvascular complications, including retinopathy, nephropathy, neuropathy, cancer and/or mortality due to cancer. A 1% decrease in the level of HbA1c has been associated with a 21% decrease in deaths tied to diabetes, a 14% decrease in myocardial infarction and a 37% decrease in microvascular complications.

Furthermore, the concentrate reduces the increase in plasma TBARS, measured in the diabetic rats treated with STZ, by 20-25%, demonstrating in vivo the antioxidant effects of these natural compounds.

The concentrate has properties favoring the reduction of lipid compounds in diabetic animals. In fact, increases of about 2 times in TC and about 4 times in TG were observed in the serum of diabetic rats, whereas the diabetic rats treated with the concentrate showed levels reduced by 40 and 24% respectively. Therefore, the data strongly suggest that treatment with the concentrate is capable of reducing the atherogenic complications correlated with diabetes.

Finally, an assessment was made of both the motor and sensory nerve conduction velocity, which is a standard endpoint for assessing the onset and progression of diabetic neuropathy. The data demonstrated that the concentrate countered, to a significant degree, the axonal degeneration caused by diabetes. The thermal and mechanical nociceptive thresholds were more than doubled in diabetic animals compared to non-diabetic animals, and the concentrate reduced the impairment by 15% and 66%, respectively, demonstrating indirectly a profound neuroprotective effect on the function of nerve fibers.

In conclusion, the data reported above clearly demonstrate the potential therapeutic effects of the polyphenolic concentrate on long-term diabetic complications. In fact, dietary supplementation with the concentrate of the present invention in a well-established model of diabetes in rats brought about the regression of various pathological endpoints.

1. A concentrate of vegetation waters and/or olive pomace comprising hydroxytyrosol and 3,4-DHPA-EDA for use in the treatment and/or prevention of diabetes, reduction of symptoms and/or side effects associated with/caused by diabetes or a pathology/complication associated with/caused by diabetes.

2. The concentrate of claim 1, wherein 3,4-DHPA-EDA is present in an amount from 0.5 to 8 g/L, and/or tyrosol is present in an amount from 0.1 to 0.4 g/L.

3. The concentrate of claim 1, wherein said concentrate further comprises:
at least one phenolic compound selected from the group consisitng of tyrosol, chlorogenic acid, β-hydroxyverbascoide, rutin, verbascoide, and luteolin; and/or
at least one metal selected from the group consisitng of sodium, calcium, magnesium and potassium; and/or
at least an anion selected from the group consisitng of chlorides, sulphates, phosphates and nitrates; and/or
at least one carbohydrate from the group consisitng of glucose, fructose, mannitol and sucrose; and/or
nitrogen.

4. The concentrate of claim 1, wherein said concentrate is obtained by means of a process comprising the steps of:
(i) microfiltering a sample of the vegetation water and/or olive pomace so as to obtain a concentrate and a permeate of microfiltration; and
(ii) concentrating by reverse osmosis the microfiltration permeate obtained in step (i).

5. The concentrate of claim 4, wherein the microfiltration step involves the use of at least one ceramic membrane, characterized by a tubular shape, and made of aluminum oxide and zirconia.

6. The concentrate of claim 4, wherein the reverse osmosis is performed by using a polymeric membrane, said membrane (1) characterized by a spiral shape and/or (2) made of a material comprising polyamide.

7. The concentrate of claim 1, formulated for oral administration.

8. The concentrate of claim 1, formulated as lozenges, pills, capsules, tablets or the like.

9. The concentrate of claim 1, formulated for topical application as a cream, oil, ointment, aerosol, gel, vaginal suppository, spray, solution, patch, gauze, bandage, granules or powder.

10. The concentrate of claim 9, further comprising agents/molecules with a biologically active function selected from the group consisting of cicatrizing, anti-inflammatory, antibiotic, emollient, soothing, analgesic and combinations thereof.

11. The concentrate of claim 1, wherein said pathology/complication associated with/caused by diabetes is selected from the group consisting of diabetic neuropathy, nephropathy, alteration of pancreatic function/structure, retinopathy, diabetic foot, and dry skin with vasomotor disautonomic manifestations.

12. The concentrate and/or the composition for use according to claim 1, wherein said symptoms and/or said side effects are selected from the group consisting of reduction of hyperglycemia, elevated levels of glycated hemoglobin, onset or development/progression of cataracts and retinopathy, and alterations in thermal and mechanical sensitivity.

13. The concentrate and/or the composition for use according to claim 1, in combination with other substances, compounds, drugs or compositions having protective and/or curative effects on diabetes, selected from the group consisting of insulin, oral hypoglycemic agents, and other substances with known anti-diabetic pharmacological activity.

14. The concentrate of claim 2, wherein 3,4-DHPA-EDA is present in an amount from 1 to 6 g/L, and/or tyrosol is present in an amount from 0.15 to 0.25 g/L.

15. The concentrate of claim 14, wherein 3,4-DHPA-EDA is present in an amount from 1.5 to 2.5 g/L.

16. The concentrate of claim 7, wherein the oral formulation is a drink.

17. The concentrate of claim 16, wherein the drink is a fruit drink.

18. The concentrate of claim 17, wherein the fruit drink is a grape juice
